(19) United States
(12) Patent Application Publication
(10) Pub. No.: US 2008/0038320 A1
(43) Pub. Date: Feb. 14, 2008

(54) COMPOSITIONS AND METHODS FOR ENHANCING VASODILATION

(75) Inventors: Bonney Oommen, Salt Lake City, UT (US); Brent L. Petersen, Twin Falls, ID (US); Loren S. Ward, Twin Falls, ID (US); Eric D. Bastian, Twin Falls, ID (US); Stanley A. Wrobel, Twin Falls, ID (US); Jessica A. Marshall, Twin Falls, ID (US)

Correspondence Address: BANNER & WITCOFF, LTD., TEN SOUTH WACKER DRIVE, SUITE 3000, CHICAGO, IL, 60606, US

(73) Assignee: Glanbia Nutritionals (Ireland) Limited, Kilkenny (IE)

(21) Appl. No.: 11/688,731

(22) Filed: Mar. 20, 2007

Related U.S. Application Data
(60) Provisional application No. 60/743,579, filed on Mar. 20, 2006.

(51) Int. Cl. A61K 38/43; A61K 38/00; A61K 9/14; A61P 43/00

(52) U.S. Cl. 424/439; 424/489; 424/94.1; 514/2

(57) ABSTRACT

Compositions and methods to achieve vasodilation through nitric oxide production pathways and Angiotensin II inhibition pathways using bovine lactoferrin and whey derived angiotensin converting enzyme inhibiting peptides are provided. Other nitric oxide ingredients may be added as well for sports nutrition applications. Such compositions and methods achieve a “pump”, which is increased blood flow and blood engorging the muscles.

This application claims priority to provisional U.S. Application No. 60/743,579, filed Mar. 20, 2006, incorporated herein by reference in its entirety.

### FIELD OF THE INVENTION

The invention relates generally to compositions and methods for stimulating vasodilation in mammals. More specifically, aspects of the invention provide compositions to stimulate vasodilation and enhance muscle mass and strength in individuals, as well as methods of stimulating vasodilation and increasing muscle mass and strength by using such dietary supplement compositions. In certain aspects, the methods and compositions are particularly suitable for use by athletes.

### BACKGROUND

A goal of many athletes and other body-conscious individuals is to maximize workouts and attain peak physical condition. Although steroids and other drugs have been used for this purpose, they have been associated with potentially harmful side effects. As a result, use of steroids and other drugs is disfavored and, in most instances, is even considered illegal.

Within especially the bodybuilding community, the “Pump” is a term that describes a feeling bodybuilders may experience when working out. This feeling, which results from blood engorging the muscles being worked, may be experienced during a workout, as well as for some period after the completion of a workout. The more strenuous the workout, the greater the resulting “Pump” would be expected. The “Pump” is perceived by some as leading to larger increases in muscle mass and strength from a workout. Thus, increased flow of blood through the veins may be desired in connection with a workout.

Certain nutritional compositions include nitric oxide or substances that increase or sustain the concentration of nitric oxide in the body. Nitric oxide is believed to have various functionalities in tissues and systems within the body including vascular tone, blood pressure, muscle contraction, muscle growth and response to contractile activity. (Sessa, W. C., et al., “Regulation of endothelial derived nitric oxide in health and disease,” Mem. Inst. Oswaldo Cruz, 2005, 100: p. 15-18; Smith, L. W., et al., “Involvement of nitric oxide synthase in skeletal muscle adaptation to chronic overload,” J. Appl. Physiol., 2002, 92: p. 2005-2011.) Increasing nitric oxide production within the endothelium of vascular tissue may cause vasodilation and thereby may enhance muscle growth.

In the area of sports nutrition, nutritional compositions may include a source of the amino acid L-arginine or similar substances. It is believed that L-arginine is directly converted to nitric oxide in the body via the nitric oxide synthase (“NOS”) family of enzymes. (Boger, R. H., et al., “Clinical Pharmacology of L-Arginine,” Ann. Rev. Pharmacol. Toxicol., 2001, 41: p. 79-99.) Many nitric oxide sports nutrition products thus include an arginine ingredient, typically arginine alpha-ketogluterate. The nitric oxide produced from arginine may stimulate vasodilation.

Nitric oxide is an endogenously produced gaseous signaling molecule in the body. It is unique in its ability to be produced by one cell, then penetrate cellular membranes and regulate the functioning of other cells. Nitric oxide is typically formed from the amino acid precursor L-arginine by the enzyme system that is known as nitric oxide synthase (“NOS”).

Nitric oxide possesses many functions within the body's tissues and systems including a role in vascular tone, blood pressure management, muscle contraction, muscle growth and cell kinetics (response to contractile activity). Endothelial cells also contribute in the maintenance of vascular tone and structure. Nitric oxide, one of the major vaso-active mediators in endothelial tissue, typically increases nitric oxide production within the endothelium of vascular tissue causing vasodilation (or vaso-relaxation) thereby increasing the “Pump” effect. Within the sports nutrition community, in particular bodybuilding, greater blood flow equals greater “Pump.”

It would therefore be desirable to identify alternative nutritional compositions that will enhance an individual's workout regimen and assist the individual in achieving his or her desired muscle composition and level of physical performance. It would also be desirable to provide methods of maximizing physical performance by, among other mechanisms, stimulating or enhancing nitric oxide production and vasodilation, as well as maximizing muscle mass and strength.

### SUMMARY

Aspects of the invention relate to dietary compositions and supplements that will enhance vasodilation effects in the body and enhance muscle mass and strength in an individual. The compositions include protein, particularly lactoferrin, angiotensin converting enzyme inhibiting peptides, and combinations thereof. In certain aspects the protein and peptides are derived from whey.

Aspects of the invention also relate to methods of administering such compositions and supplementing an individual's diet to stimulate nitric oxide production, promote vasodilation, enhance the growth of lean muscle mass, and enhance muscle strength. In certain aspects, the compositions are administered to athletes. For example, the compositions may be administered to bodybuilders. In certain aspects, the methods include administering effective amounts of compositions to stimulate vasodilation and achieve an increase in muscle mass and strength.

Aspects of the invention also relate to compositions that include lactoferrin and angiotensin converting enzyme inhibiting peptides in amounts effective for treating, maintaining or otherwise affecting vascular systems in an animal, such as a person. Methods of administering the compositions and of supplementing a diet with such compositions are provided for, among other purposes, increasing blood flow and improving circulation, lowering blood pressure and reducing hypertension.

### BRIEF DESCRIPTION OF THE DRAWINGS

A more complete understanding of the present invention and the advantages thereof may be obtained with reference to the following description and accompanying drawings and wherein:

FIG. 1 illustrates a pathway for vasodilation in accordance with an aspect of the invention;

FIG. 2 illustrates a pathway for vasoconstriction which can be inhibited in accordance with another aspect of the invention;

FIG. 3 illustrates another pathway for vasodilation in accordance with another aspect of the invention;

FIG. 4 illustrates another pathway for vasoconstriction which can be inhibited in accordance with another aspect of the invention;

FIG. 5 is a graphical representation illustrating the effect of lactoferrin on nitric oxide production in HPAE-26 cells;

FIG. 6 is a graphical representation illustrating the effect of angiotensin converting enzyme inhibiting peptides on nitric oxide production in HPAE-26 cells; and

FIG. 7 is a graphical representation illustrating the effect on a formulation in accordance with the present invention on nitric oxide production in HPAE-26 cells.

### DETAILED DESCRIPTION

Compositions in accordance with the invention include lactoferrin, nitric oxide synthase (“NOS”) peptides and/or angiotensin converting enzyme inhibiting peptides (“ACE inhibiting peptides”), and combinations thereof. NOS peptides are those peptides that will increase the nitric oxide production in endothelial cells. These compositions promote vasodilation, enhance circulation, and provide sports nutrition solutions. These compositions may be used independently or in conjunction with other nutritional supplements, such as sports nutrition products, including nitric oxide products. While the compositions are described with respect to ACE inhibiting peptides, it is contemplated that any NOS peptides and/or ACE inhibiting peptides could be utilized.

Nitric oxide production is of interest, for example, in sports nutrition generally and other fitness and health applications. It is also of interest for increasing energy levels, providing positive effects on heart health, treating the effects of diabetes. The compositions may be used to provide beneficial effects in these and other applications.

The dietary compositions and methods in accordance with aspects of the invention may provide increased muscle mass and strength, as well as enhanced vasodilation effects in individuals. In one aspect, the compositions and methods may be used by athletes, including bodybuilders. Use of the compositions and methods is not intended to be limited to athletes, however, but is intended for any individual, as well as for all animals.

Sources of lactoferrin include, but are not intended to be limited to, mammalian lactoferrin (e.g., bovine and human), recombinant lactoferrin, N-terminal variant lactoferrin, lactoferrin hydrolysates, lactoferrin lacking glycosylates, lactoferrin glycosylation variants, apolactoferrin (lactoferrin without bound iron), lactoferrin bound to other metals (e.g., zinc, copper, manganese, selenium), and combinations thereof. It is contemplated that other sources and equivalents also could be used. In certain aspects, the lactoferrin is derived from whey.

Peptides, including NOS peptides and ACE inhibiting peptides, used in the inventive compositions may be derived from any food protein, including but not limited to, fish protein, rice protein, soy protein, casein, egg, and any other suitable source of food-grade protein. It is contemplated that other sources and equivalents also could be used. In certain aspects, the protein is whey protein, including hydrolyzed whey protein, or derivatives thereof. Isolated peptides or blends of peptides having a range of molecular weights may be used.

The compositions also may include other components, such as nitric oxide, sources of nitric oxide, vitamins, minerals, caffeine, herbal substances, or other nutritional ingredients. The compositions further may include flavorings, colorants, or any other desired additives in accordance with techniques known to persons skilled in the art.

The dietary compositions may be provided in various forms, including gel, solid or liquid forms. The compositions can be administered directly, mixed with other solids or liquids, or incorporated into any number of solid and liquid food products. For example, the compositions can be formed as gels, tablets, chewable tablets, capsules, and liquid syrup to be administered directly to an individual. The compositions also can be incorporated into liquid beverages or mixed with liquid beverages by the consumer. Liquid beverages include water, acidic juice beverages (e.g., orange juice, apple juice, grape juice, grapefruit juice, cranberry juice, or blended juices), acidic beverages (e.g., sport beverages, Gatorade®), neutral pH beverages (e.g., milk UHT dairy, RTD nutritional, soy milk, or shakes and other blended beverages such as milkshakes, smoothies, frappes). The compositions also can be incorporated into nutritional supplement foodstuffs (e.g., gel packs, puddings, cereals, energy bars or other health bars), confectionery products (e.g., chews or chewing gum,), and dairy products (e.g., yogurt, cheese, or processed cheese).

The dietary compositions may be prepared using any conventional processing techniques, such as for example spray drying or filtration. Filtration methods may involve microfiltration, ultrafiltration, nanofiltration, reverse osmosis and/or combinations of such methods. Various chromatography separation techniques may be used including but not limited to anionic and cationic exchange, hydrophobic resins, affinity chromatography in order to separate proteins and peptides.

Without wishing to be bound by any theory, it is believed that milk contains bioactive components that promote health and survival. Whey proteins, a fraction of dairy proteins, may possess the highest concentration of bioactivity.

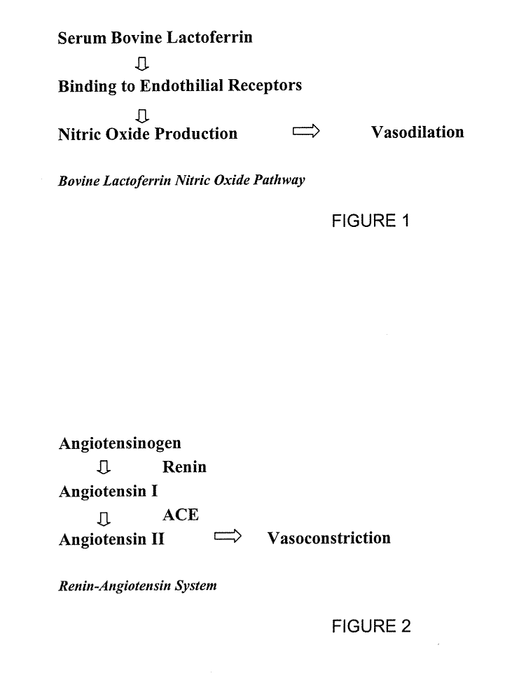

Without wishing to be bound by any theory, it is believed that lactoferrin has bioactivity regarding nitric oxide within the body. Lactoferrin is a whey protein, that is, a bioactive whey peptide. Lactoferrin, which may be present in only trace amounts in many sources of whey, can be isolated and concentrated. Referring to FIG. 1, lactoferrin may bind to epithelial tissue within the vascular system, causing vasodilation via a nitric oxide mechanism. The lactoferrin binds to endothelial receptors, which enhances nitric oxide production. It is believed that vasodilation may act in a dose response manner, increasing as the concentration of lactoferrin increases.

Without wishing to be bound by any theory, it is also believed that whey derived NOS peptides and/or ACE inhibiting peptides may possess bioactivity, causing vasodilation via any one of three mechanisms. The three mechanisms relate to the renin-angiotensin system, ACE enzyme and bradykinin and ACE enzyme and endothelin I as described in more detail below. ACE is involved in vascular action within the body through numerous mechanisms. By inhibiting the action of ACE, vasodilation may be stimulated or greater vasodilation may be achieved.

Referring to FIG. 2, a first pathway for ACE-related vasodilation relates to the renin-angiotensin system, which involves the conversion of angiotensinogen by the enzyme renin into angiotensin I. Angiotensin I is converted into a potent vasoconstrictor, angiotensin II, by the enzyme ACE. ACE inhibiting peptides, such as those commercially available from Glanbia Nutritionals, Monroe, Wis., may inhibit the enzyme ACE. By inhibiting ACE, it may be possible to inhibit the conversion of angiotensin I into angiotensin II. This in turn would keep vascular tissue dilated by inhibiting vasoconstriction.

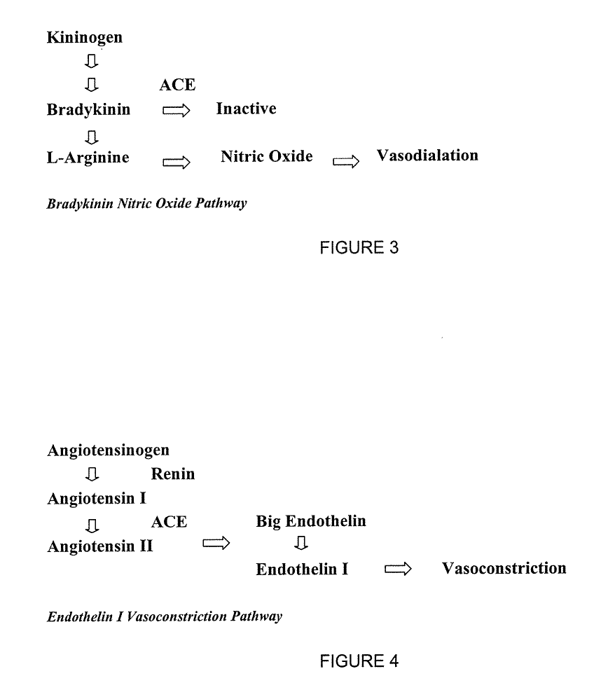

Referring to FIG. 3, a second pathway for vasodilation relates to the enzyme ACE and bradykinin. Bradykinin has an effect on nitric oxide, acting as a potential stimulator for production of nitric oxide from arginine. Kininogen is converted to bradykinin through a series of enzymatic steps. Bradykinin is a very bioactive compound which can bind to endothelial beta receptors and cause vasodilation through the production of nitric oxide from L-arginine. The alternative would be inactivation of bradykinin through proteolysis by the enzyme ACE.

Referring to FIG. 4, a third pathway relates to the vasoconstriction compound endothelin I and the ACE enzyme. Angiotensin I is converted to angiotensin II by ACE. Angiotensin II results in vasoconstriction and has other functions. Angiotensin II stimulates the vascular endothelium to produce the vasoconstrictor endothelin-I from big endothelin. Inhibiting ACE from converting angiotensin I into angiotensin II may cause a vasodilation effect by limiting the formation of endothelin-I.

According to one aspect of the invention, dietary compositions and supplements that enhance vasodilation include lactoferrin or a source of lactoferrin. The compositions and supplements may act through a pathway of producing nitric oxide from lactoferrin. The lactoferrin binds to epithelial tissue within the vascular system, stimulating production of nitric oxide.

In another aspect of the invention, dietary compositions and supplements that enhance vasodilation include ACE inhibiting peptides or a source of ACE inhibiting peptides. The compositions and supplements may act by inhibiting the activity of the enzyme ACE to avoid possible mechanisms relating to vasoconstriction.

Compositions and dietary supplements in accordance with various aspects of the invention deliver effective amounts of lactoferrin, NOS peptides and/or ACE inhibiting peptides to an individual. The compositions generally include lactoferrin in an amount between about 0.01 grams and about 100 grams. Amounts less than 0.01 grams or greater than 100 grams may also be possible depending on the formulation. In other aspects, the amount of lactoferrin is about 2 grams or less, about 10 grams or less, or about 100 grams or less. The compositions generally include NOS and/or ACE inhibiting peptides in an amount between about 0.01 grams and about 1000 grams. Amounts less than 0.01 grams or greater than 1000 grams may also be possible depending on the formulation. In other aspects, the amount of peptides is about 20 grams or less, about 200 grams or less, or about 1000 grams or less. In certain aspects, the relative amounts of lactoferrin and peptides in the compositions may range from about 0.1 grams of lactoferrin to about 20 grams of peptides up to about 20 grams of lactoferrin to about 0.01 grams of peptides. Larger or smaller ratios also are contemplated.

According to another aspect of the invention, dietary compositions and supplements including lactoferrin, NOS peptides and/or ACE inhibiting peptides are administered to individuals. In certain aspects, effective amounts of the compositions are administered to athletes, including bodybuilders, in connection with an exercise program. The compositions are administered before, during or after exercise to enhance the effects of the exercise. Thus, methods of enhancing vasodilation and methods of increasing muscle size and strength also are provided. Methods of supplementing an individual's diet to enhance the effects of exercise also are provided.

Administration of the compositions to an individual will vary depending on the desired effect. An effective amount may be determined by a person of skill in the art using the teachings herein and knowledge in the art. The compositions may be administered on a daily basis and may be administered one or more times per day. One or more servings are administered each time to provide an effective amount of lactoferrin, NOS peptides and/or ACE inhibiting peptides. An “effective amount,” as used herein means an amount, administered at dosages and for a time, effective for achieving desired results. Thus, an effective amount may vary depending on various factors, including but not limited to, gender, age, body type, weight and the desired effect.

Compositions and methods in accordance with aspects of the invention also may be used in connection with other vascular-related mechanisms and effects, including those described in any of the references identified herein. For example, the compositions and methods can be used to regulate vascular tone or otherwise in the treatment, prevention or maintenance of vascular conditions, such as to increase blood flow to improve circulation, lower blood pressure and reduce hypertension.

### EXAMPLE 1

In this example, an all natural whey derived nitric oxide synthase activator was developed in a formulation as set forth in Table 1.1.

TABLE 1.1

| | |
|---|---|
| Protein, dry basis | 90.0% |
| Fat | 0.5% |
| Minerals | 4.0% |
| Lactose | 1.0% |
| Moisture | 5.0% |
| pH (10% at 20° C.) | 7.2 to 7.8 |

The formulation exhibited the amino acid breakdown set forth in Table 1.2.

TABLE 1.2

| | g/100 g |
|---|---|
| Amino Acid | protein |
| Aspartic Acid | 11.6 |
| Threonine | 5.9 |
| Serine | 4.9 |
| Glutamic Acid | 14.6 |
| Glycine | 3.2 |
| Alanine | 6.1 |
| Valine | 5.6 |
| Isoleucine | 4.0 |
| Leucine | 9.8 |
| Tyrosine | 3.7 |
| Phenylanaline | 4.2 |
| Histidine | 1.8 |
| Lysine | 9.0 |
| Arginine | 5.0 |
| Proline | 5.1 |
| Cystine | 3.3 |
| Methionine | 1.2 |
| Tryptophan | 2.4 |

The formulation exhibited nutritional characteristics as set forth in Table 1.3.

TABLE 1.3

| Calories | 380 | |
|---|---|---|
| Calories from Fat | 2 | |
| Total Fat | 0.18 | g |
| Saturated Fat | 0.09 | g |
| Polyunsaturated Fat | 0.01 | g |
| Monounsaturated Fat | 0.08 | g |
| Trans Fatty Acid | — | |
| Cholesterol | 0.8 | mg |
| Total Carbohydrate | 4.2 | g |
| Dietary Fiber | — | |
| Sugars | 2.7 | g |
| Protein | 90 | g |
| Vitamin A | — | |
| Vitamin C | — | |
| Thiamin | — | |
| Niacin | — | |
| Riboflavin | — | |
| Calcium | 240 | mg |
| Sodium | 530 | mg |
| Potassium | 160 | mg |
| Magnesium | 60 | mg |
| Iron | 10 | mg |
| Phosphorus | 100 | mg |

This example illustrates a formulation utilizing a combination of lactoferrin and nitric oxide synthase peptides with angiotensin converting enzyme inhibiting peptides. The composition included about a 60% lactoferrin component and about 40% of a nitric oxide synthase peptide component, which was substantially all angiotensin converting enzyme inhibiting peptides. The formulation promoted the vasodilation and vasoconstriction as described above.

### EXAMPLE 2

In this example, another formulation of an all natural whey derived nitric oxide synthase activator was developed as set forth in Table 2.1

TABLE 2.1

| | |
|---|---|
| Protein, dry basis | 90.0% |
| Fat | 1.0% |
| Minerals | 7.0% |
| Lactose | 1.5% |
| Moisture | 5.0% |
| pH (10% at 20° C.) | 7.2 to 7.8 |

The formulation exhibited the amino acid breakdown set forth in Table 2.2.

TABLE 2.2

| | g/100 g |
|---|---|
| Amino Acid | protein |
| Aspartic Acid | 11.9 |
| Threonine | 7.0 |
| Serine | 4.6 |
| Glutamic Acid | 19.0 |
| Glycine | 1.6 |
| Alanine | 4.9 |
| Valine | 5.6 |
| Isoleucine | 6.4 |
| Leucine | 10.0 |
| Tyrosine | 2.7 |
| Phenylanaline | 2.9 |
| Histidine | 1.6 |
| Lysine | 8.8 |
| Arginine | 1.3 |
| Proline | 5.9 |
| Cystine | 2.2 |
| Methionine | 1.8 |
| Tryptophan | 2.0 |

The formulation exhibited nutritional characteristics as set forth in Table 2.3.

TABLE 2.3

| Calories | 370 | |
|---|---|---|
| Calories from Fat | 4 | |
| Total Fat | 0.42 | g |
| Saturated Fat | 0.25 | g |
| Polyunsaturated Fat | 0.03 | g |
| Monounsaturated Fat | 0.10 | g |
| Trans Fatty Acid | 0.02 | g |
| Cholesterol | 2 | mg |
| Total Carbohydrate | 1.6 | g |
| Dietary Fiber | — | |
| Sugars | 0.7 | g |
| Protein | 88 | g |
| Vitamin A | — | |
| Vitamin C | — | |
| Thiamin | — | |
| Niacin | — | |
| Riboflavin | — | |
| Calcium | 500 | mg |
| Sodium | 1,200 | mg |
| Potassium | 400 | mg |
| Magnesium | 130 | mg |
| Iron | 0.3 | mg |
| Phosphorus | 320 | mg |

This example illustrates a formulation utilizing a combination of lactoferrin and nitric oxide synthase peptides with angiotensin converting enzyme inhibiting peptides. The composition included about a 0.3% lactoferrin component and about 99.7% of a nitric oxide synthase peptide component, which was substantially all angiotensin converting enzyme inhibiting peptides. The formulation promoted the vasodilation and vasoconstriction as described above.

### EXAMPLE 3

This example illustrates the potency and the role of both low and high molecular weight fractions. Formulations were separated into two fractions: Fraction A based on high molecular size and Fraction B based on low molecular size. The effect of each fraction separately and the combined formulation (a combination of both A and B) on NO production in human pulmonary artery endothelial (“HPAE”) cells was studied. Fraction A represented the use of the lactoferrin mechanism described above. Fraction B represented the use of an ACE inhibiting peptide mechanism as described above.

HPAE-26 cells were grown to confluence and collected. The cells were suspended in appropriate media and the cell count was adjusted to 1×10⁵ ml⁻¹. The cells (100 μl well⁻¹; 1×10⁴) were plated in 96-well, flat bottom plates. Granulocyte Macrophage Colony Stimulating Factor (GM-CSF) (10 ng ml⁻¹ in 50 μl well⁻¹) was added to all the wells except the media control.

Fractions A and B were dissolved in HPAE-26 cell culture media and diluted using the same media so as to result in appropriate concentrations in each well. The components at the appropriate dilutions (50 μl well⁻¹) were added to the wells to get a final volume of 200 μl well⁻¹, resulting in the final concentrations. The final concentrations of the components were 37.5, 75, and 112.5 μM for Fraction A and 2, 4, and 6 mM for Fraction B. Tests were also performed on the formulation (combination of fraction A and B) at 2.1, 4.1, and 6.1 mM concentrations. The molar ratios of the combined fractions (A:B) were 0.0375:2, 0.075:2, 0.1125:4, 0.0375:4, 0.075:4, 0.1125:4, 0.0375:6, 0.075:6 and 0.1125:6.

The plates were incubated at 37° C. and 5% CO₂ for 24 hours. After incubation, the supernatants were collected and tested for nitrate to measure nitric oxide synthesis. Data was analyzed using analysis of variance.

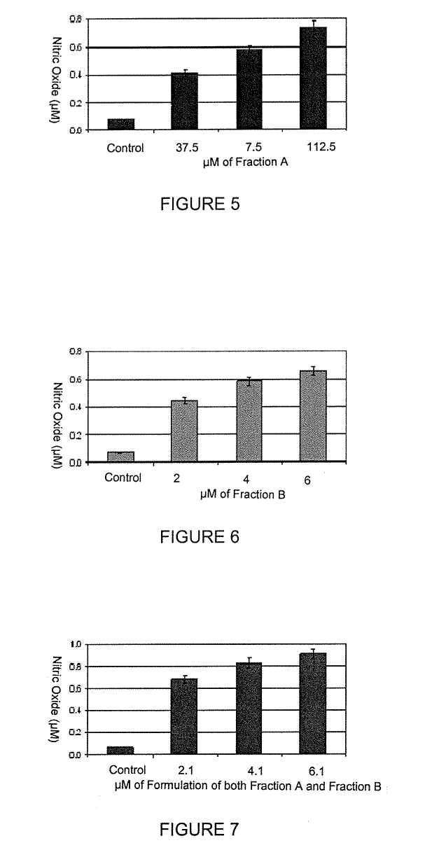

The control (no added test material) inherently produced 0.07 μM of nitric oxide. Fraction A increased nitric oxide production significantly (P<0.05) in HPAE cells as seen in FIG. 5. The maximum increase in nitric oxide production was 10.57 times the nitric oxide production of the control. The nitric oxide production increased to 0.41, 0.58, and 0.74 μM as the concentration of Fraction A increased. Nitric oxide production increased between about 474 to about 938% compared to the control. These percentages are calculated based on the difference between nitric oxide production in the treatment and nitric oxide production in the control. The difference is compared to the nitric oxide production in the control in order to obtain the percentage increase in nitric oxide production.

A similar result was seen with Fraction B (FIG. 6). As the concentration of fraction B increased from 2 mM to 6 mM, nitric oxide production increased significantly (P<0.05) from 0.45 to 0.66 μM. The maximum increase in nitric oxide production shown was 10.4 times compared to the control.

FIG. 7 portrays the effect of the formulation (containing both fractions A and B) on nitric oxide production. The formulation may provide an interaction effect between fractions A and B. As the formulation concentration increased from 0 to 2.1 mM, the nitric oxide production increased 855% as compared to the control. The maximum increase in nitric oxide production shown was 1169% . Similarly at 4.1 and 6.1 mM, the percentage increase from the control with no formulation was 1053 and 1169% respectively. The nitric oxide production for the control as about 0.0716 micromoles, whereas the 2.1, 4.1 and 6.1 mM formulations yielded 0.6833, 0.8250 and 0.9083 mM. The formulation included peptides with the following molecular weights:

| Peptide | Molecular weight | | |
|---|---|---|---|
| 7% | >16000 | 1120 | 16000 |
| 41% | 7000-16000 | 4715 | 11500 |
| 18% | 5000-7000 | 1080 | 6000 |
| 13% | 1000-5000 | 390 | 3000 |
| 22% | <1000 | 220 | 1000 |
| | Average peptide length | 7525 | |

The effectiveness of the fractions, or NOS activators, present in the formulation augments the nitric oxide production without addition of external arginine. This suggests that the NOS activators boost nitric oxide production by factors not related to arginine, Ca⁺⁺, or bradykinin. Without wishing to be bound, it is believed that the NOS activators may increase the transcription of NOS gene and contribute to reducing the negative feedback mechanism on nitric oxide production.

The experimental formulations boosted nitric oxide production by up to about 9.5 times at the minimal concentrations tested in human endothelial cells. Size-based fractions of the formulation are critical components and act synergistically to yield such an increase in nitric oxide production. Thus, the NOS activators in the formulation appear to play a role is altering the regulatory mechanisms involved with nitric oxide production in human endothelial cells. These cells are critical in producing nitric oxide, which increases blood flow and “Pump”, thereby delivering vital nutrients to exercising muscle.

The present invention includes any novel feature or combination of features disclosed herein either explicitly or any generalization thereof. Although the invention has been described with respect to specific examples including presently preferred modes of carrying out the invention, those skilled in the art will appreciate that there are numerous variations and permutations of the above described compositions and techniques. Thus, the spirit and scope of the invention should be construed broadly.

What is claimed is:

1. A dietary composition comprising:
a first component including lactoferrin;
a second component including angiotensin converting enzyme inhibiting peptides;
the components included in amounts effective for stimulating vasodilation in an individual.

2. The composition in claim 1 wherein the composition is a powder that can be added to a food product.

3. The composition in claim 1 wherein the composition is a liquid that can be added to a food product.

4. The composition of claim 1 wherein the composition is a liquid that is ready to drink.

5. The composition of claim 2 wherein the food product is an energy bar or food bar.

6. The composition of claim 1 wherein the composition is a gel.

7. The composition of claim 4 wherein the liquid is selected from the group consisting of water, acidic juice beverages, sport beverages and neutral pH beverages.

8. The composition of claim 1 wherein the composition also includes at least one vitamin, mineral, nitric oxide, caffeine, herbal substance, nutritional ingredients or combinations thereof.

9. The composition of claim 1 wherein the first component includes between about 0.01 grams and about 100 grams of lactoferrin.

10. The composition of claim 1 wherein the second component includes between about 0.01 and 1000 grams of angiotensin converting enzyme inhibiting peptides.

11. The composition of claim 1 wherein the first component includes between about 0.01 grams and about 100 grams of lactoferrin and the second component includes less than about 1000 grams of angiotensin converting enzyme inhibiting peptides.

12. The composition of claim 1 wherein the components are included in amounts effective for stimulating the growth of muscle mass.

13. The composition of claim 1 wherein the components are included in amounts effective for regulating vascular tone.

14. The composition of claim 1 wherein the components are included in amounts effective for enhancing muscle strength.

15. The composition of claim 1 wherein the composition is effective for humans.

16. The composition of claim 1 wherein the composition is effective for animals.

17. A method for enhancing vasodilation in an individual comprising:
ingesting a composition prior to exercise, the composition including lactoferrin and angiotensin converting enzyme inhibiting peptides in an amount effective for enhancing the effect of vasodilation resulting from exercise.

18. The method of claim 17 wherein the composition promotes inhibition of vasoconstriction.

19. The method of claim 17 wherein the composition is a gel.

20. The method of claim 17 wherein the composition is a particulate.

21. A workout performance enhancing composition comprising:
a component including nitric oxide synthase peptides;
the component provided in an amount effective for stimulating vasodilation in an individual.

22. The composition of claim 21 wherein the component includes angiotensin converting enzyme inhibiting peptides.

23. The composition of claim 22, wherein the component includes a lactoferrin.

24. The composition of claim 21 wherein the composition also includes at least one vitamin, mineral, nitric oxide, caffeine, herbal substance, nutritional ingredients or combinations thereof.

25. The composition of claim 21 wherein the component is included in an amount effective for stimulating the growth of muscle mass.

26. The composition of claim 21 wherein the component is included in an amount effective for regulating vascular tone.

27. The composition of claim 21 wherein the component is included in an amount effective for enhancing muscle strength.
